(19) United States
(12) Patent Application Publication
(10) Pub. No.: US 2021/0161986 A1
(43) Pub. Date: Jun. 3, 2021

(54) CAFFEINE COMPOSITIONS AND METHODS OF USE

(71) Applicant: BARISTA MIST PTY LTD, Kalinga (AU)

(72) Inventors: Traian Vasile Chirila, Robertson (AU); Tai Mountford Smith, Kalinga (AU); Shuko Suzuki, Ormeau (AU)

(21) Appl. No.: 17/048,551

(22) PCT Filed: Apr. 15, 2019
(86) PCT No.: PCT/AU2019/050336

§ 371 Date: Oct. 16, 2020

(30) Foreign Application Priority Data
Apr. 16, 2018 (AU) 2018901266

(51) Int. Cl. A61K 36/74; A61K 9/00; A61K 9/08; A61K 31/522; A61K 47/38; A61K 47/12; A61P 25/30; A61K 47/10

(52) U.S. Cl. CPC A61K 36/74; A61K 9/0043; A61K 9/08; A61K 31/522; A61K 47/38; A61K 2236/331; A61P 25/30; A61K 47/10; A61K 2236/15; A61K 2236/37; A61K 47/12

(57) ABSTRACT

A pharmaceutical composition formulated for intranasal administration comprising a natural caffeine extract, a pharmaceutically acceptable excipient, a pharmaceutically acceptable aqueous carrier; and, optionally, additional caffeine, or a pharmaceutically acceptable salt, solvate, derivative or metabolite thereof. Methods of preparation and methods of use are also described.

This application claims priority to Australian Provisional Application No. 2018901266 entitled “Compositions and Methods” filed on 16 Apr. 2018, the entire content of which is hereby incorporated herein by reference in its entirety.

### FIELD OF THE INVENTION

The compositions and methods described herein relate generally to preventing, treating or alleviating caffeine withdrawal or a symptom of caffeine withdrawal or coffee craving in an individual. In some particular aspects, the compositions and methods described herein relate to preventing, treating or alleviating caffeine withdrawal or coffee craving in an individual whilst maintaining a nil-by-mouth status.

### BACKGROUND OF THE INVENTION

Brewed coffee is one of the most popular beverages in the world, being second in consumption only to water [see M. S. Butt, M. T. Sultan: “Coffee and its consumption: benefits and risks”, Critical Reviews in Food Science and Nutrition, 51 (2011) 363-373]. Research has linked coffee consumption to potential health benefits including prevention of some chronic and degenerative diseases [see E. M. Laska, A. Sunshine, F. Mueller, W. B. Elvers, C. Siegel, A. Rubin: “Caffeine as an analgesic adjuvant”, Journal of the American Medical Association, 251 (1984) 1711-1718; J. V. Higdon, B. Frei: “Coffee and health: a review of recent human research”, Critical Reviews in Food Science and Nutrition, 46 (2006) 101-123; T. P. Heffernan, M. Kawasumi, A. Blasina, K. Anderes, A. H. Conney, P. Nghiem: “ATR-Chkl pathway inhibition promotes apoptosis after UV treatment in primary human keratinocytes: potential basis for the UV protective effects of caffeine”, Journal of Investigative Dermatology, 129 (2009) 1805-1815; J. Costa, N. Lunet, C. Santos, A. Vaz-Carneiro: “Caffeine exposure and the risk of Parkinson's disease: a systematic review and meta-analysis of observational studies”, Journal of Alzheimer's Disease, 20 (2010) S221-S238; F. Song, A. A. Qureshi, J. Han: “Increased caffeine intake is associated with reduced risk of basal cell carcinoma of the skin”, Cancer Research, 72 (2012) 3282-3289; I. A. Ludwig, M. N. Clifford, M. E. J. Lean, H. Ashihara, A. Crozier: “Coffee: biochemistry and potential impact on health”, Food and Function, 5 (2014) 1695-1717; M. C. Cornelis: “Gene-coffee interactions and health”, Current Nutrition Reports, 3 (2014) 178-195]. M. B. Vieira, R. Magriço, C. V. Dias, L. Leitão, J. S., Neves: “Caffeine consumption and mortality in chronic kidney disease: a nationally representative analysis”, Nephrology Dialysis Transplantation, 2018, 1-7. DOI: 10.1093/ndt/gfy234].

The substances present in coffee can improve psychomotor performance in humans, measured by both objective and subjective criteria such as attention, auditory vigilance, arousal, interest, alertness, wakefulness, anxiety, etc. [see D. A. Sawyer, H. L. Julia, A. C. Turin: “Caffeine and human behavior: arousal, anxiety, and performance effects”, Journal of Behavioral Medicine, 5 (1982) 415-439; G. Yu, V. Maskray, S. H. D. Jackson, C. G. Swift, B. Tiplady: “A comparison of the central nervous system effects of caffeine and theophylline in elderly subjects”, British Journal of Clinical Pharmacology, 32 (1991) 341-345; T. T. Brunyé, C. R. Mahoney, H. R. Lieberman, H. A. Taylor: “Caffeine modulates attention network function”, Brain and Cognition, 72 (2010) 181-188; T. T. Brunyé, C. R. Mahoney, H. R. Lieberman, G. E. Giles, H. A. Taylor: “Acute caffeine consumption enhances the executive control of visual attention in habitual consumers”, Brain and Cognition, 74 (2010) 186-192; S. J. L. Einöther, T. Giesbrecht: “Caffeine as an attention enhancer: reviewing existing assumptions”, Psychopharmacology, 225 (2013) 251-274; J. Lanini, J. C. Fernandes Galduróz, S. Pompéia: “Acute personalized habitual caffeine doses improve attention and have selective effects when considering the fractionation of executive functions”, Human Psychopharmacology: Clinical and Experimental, 31 (2016) 29-43].

For many people, a “morning coffee” forms part of their normal daily routine. Failure to consume a cup of coffee can result in the distraction of coffee cravings, sometimes acute; or symptoms of caffeine withdrawal. Without a regular dose of caffeine, a pharmacologically active drug substance present in coffee, some people may feel sluggish and fatigued. In some cases, headache and mood disturbance can arise. These are typical symptoms of caffeine withdrawal.

Caffeine is an antagonist of the adenosine receptors in humans, and its absence may cause migraines due to intracranial vasodilatation [see R. Guieu, C. Devaux, H. Henry: “Adenosine and migraine”, The Canadian Journal of Neurological Sciences, 25 (1998) 55-58]. When associated with reduced catecholamine and serotonin activity, the absence of caffeine may also lead to fatigue, mild depression and impaired cognitive function [see L. M. Juliano, R. R. Griffiths: “A critical review of caffeine withdrawal: empirical validation of symptoms and signs, incidence, severity, and associated features”, Psychopharmacology, 176 (2004) 1-29]. A recent study [M. A. Palmer, J. D. Sauer, A. Ling, J. Riza: “Caffeine cravings impair memory and metacognition”, Memory, 25 (2017) 1225-1234] found that caffeine cravings can be similar to the effects of heroin or cocaine withdrawal, and that missing the first coffee of the day can impair memory performance, resolution (i.e. ability to distinguish items that would be remembered from those that would not), and calibration (i.e. correspondence between predicted and actual accuracy).

Coffee craving or caffeine withdrawal is a particular problem for busy people who may lack time or opportunity to access a coffee beverage. This can result in caffeine withdrawal symptoms or coffee craving leading to impaired performance or discomfort. Caffeine withdrawal can also present a significant issue for patients required to fast in advance of a medical procedure such as anaesthesia, surgery, blood tests, colonoscopy, endoscopy, and the like, where it may be mandatory to retain a nil-by-mouth status for a period of time.

Most surgical patients in preparation for surgery are required to fast for four to six hours. The reason is to optimize safety during anaesthesia by reducing the risks and complications of aspiration of stomach contents. Although fasting protocols vary, in general a strict nil-by-mouth period of at least four hours is required.

Patients scheduled for gastrointestinal investigations such as endoscopy and colonoscopy procedures are also required to fast to ensure that their gastrointestinal tract is empty. Some radiological investigations require fasting where food residue in the gastrointestinal tract may impact negatively on the quality of the imaging of some organs such as the liver, gallbladder, spleen or pancreas. Patients requiring blood tests to check for conditions such as diabetes, or to investigate their cholesterol levels, may also be required to fast.

In such circumstances, the patient is unable to drink coffee, or any other caffeinated drink, and this may result in caffeine withdrawal symptoms or coffee craving in the patient. Delays in performing surgery or a medical procedure may further prevent patients from accessing coffee to address their cravings, thus exacerbating the withdrawal effects.

Accordingly, there is a need for alternative methods and alternative compositions for delivering caffeine to an individual that avoids oral administration. It will be appreciated that these alternative methods are not limited to use in fasting patients undergoing acute caffeine withdrawal, as they may find application in any situation where the administration of caffeine to an individual is desired or beneficial. Potential individuals who may benefit from a convenient source of caffeine may include long-distance drivers, nightshift workers, students, athletes, military personnel, weight watchers, and the like. Non-oral administration routes may also be beneficial where oral administration of coffee may stain dental enamel or reduce the effectiveness of teeth whitening procedures.

Non-oral administration routes include parenteral administration routes, such as intravascular, intracerebral, transdermal, subcutaneous, or rectal routes. However, there are drawbacks associated with these administration routes. For instance, intravenous injections or infusions are associated with potential health hazards, the need for specialized personnel and equipment, and poor patient compliance, making this route unsuitable for outpatient use in chronic therapies. In another instance, the transdermal delivery through drug-loaded patches limits the range of drugs that can be delivered to only those lipophilic and very active, and is characterized by delayed absorption of the drug due to a low permeability of the densely keratinized outermost layer of the epidermis. Furthermore, methods such as intravascular, intramuscular, intracerebral, subcutaneous, transdermal and intraocular administration are essentially medical procedures and are not considered to be widely acceptable or desirable for delivery of caffeine as they are inconvenient and are not suitable for self-administration in a variety of circumstances.

Pharmaceutical compositions for intranasal delivery comprising caffeine as an ingredient are known, and have been described in WO 2015/063239 and U.S. Pat. Nos. 4,778,810, 5,169,849 and 5,508,282. U.S. Pat. No. 4,778,810 (Nastech Pharmaceuticals Company Inc) describes nasal compositions useful for the delivery of caffeine alone, or with other therapeutic agents such as analgesics, anti-inflammatory or antipyretic agents. U.S. Pat. No. 5,169,849 (Sandoz Ltd) describes nasal pharmaceutical compositions comprising dihydroergotamine incorporating an agent such as caffeine to antagonize the ciliary function depression effect of the dihydroergotamine. WO 2015/063239 (Innotesto BVBA, Veramed B.V.) describes nasal compositions comprising caffeine and/or theobromine for cleaning nasal passages or stimulating mucociliary clearance. U.S. Pat. No. 5,508,282 (J. Tulin-Silver) describes a method for treating rhinosinusitis using nasal delivery of a composition comprising ascorbic acid and caffeine.

Known commercially available caffeine-containing compositions for nasal administration consist of caffeine in an isolated purified form. Since caffeine is a bitter tasting and odourless crystalline solid, such caffeine-containing pharmaceutical compositions for nasal administration may be considered by the user to provide an experience akin to taking medication, and thus lack the sense of satisfaction associated with the aroma and taste provided by consuming a caffeinated beverage such as coffee.

Some known nasal compositions are intended for providing a local pharmacological action and may only comprise a very low concentration of caffeine to deliver a small amount locally in the nasal cavity. These compositions are unlikely to be able to provide an adequate dose of caffeine to induce a noticeable effect on the central nervous system.

Accordingly, there is a need for a method of treating or alleviating the effects of caffeine withdrawal or coffee craving whilst maintaining a nil-by-mouth status that addresses one or more of the problems associated with known delivery methods. There is also a need for a composition for nasal administration which can address one or more of the disadvantages associated with known nasal compositions comprising caffeine.

### SUMMARY OF THE INVENTION

The inventors have discovered that caffeine withdrawal or symptoms of caffeine withdrawal and/or a craving for coffee can be alleviated by intranasal administration of a composition comprising a natural caffeine extract, preferably a coffee extract.

Accordingly, in a first aspect there is provided a method of treating or preventing a craving for coffee in an individual, or treating or preventing caffeine withdrawal or a symptom of caffeine withdrawal in an individual, comprising administering to the individual via intranasal delivery an aqueous pharmaceutical composition comprising a natural caffeine extract and at least one pharmaceutically acceptable excipient.

In another aspect, there is provided a method of treating or preventing a craving for coffee in an individual, or treating or preventing caffeine withdrawal or a symptom of caffeine withdrawal in an individual, whilst maintaining a nil-by-mouth status comprising administering to the individual via intranasal delivery an aqueous pharmaceutical composition comprising a natural caffeine extract and at least one pharmaceutically acceptable excipient.

In yet another aspect, there is provided an aqueous pharmaceutical composition comprising a natural caffeine extract and at least one pharmaceutically acceptable excipient for use in treating or preventing a craving for coffee, or treating or preventing caffeine withdrawal or a symptom of caffeine withdrawal, wherein the composition is adapted for intranasal delivery.

In a yet further aspect, there is provided a use of an aqueous pharmaceutical composition comprising a natural caffeine extract and at least one pharmaceutically acceptable excipient, in the manufacture of a medicament for treating or preventing a craving for coffee, or treating or preventing caffeine withdrawal or a symptom of caffeine withdrawal, wherein the composition is adapted for intranasal delivery.

In preferred embodiments, the natural caffeine extract is coffee extract.

In preferred embodiments, the methods or uses of the invention maintain a nil-by-mouth status.

In preferred embodiments, the composition also comprises additional caffeine or a pharmaceutically acceptable salt, derivative, metabolite or solvate thereof to supplement the amount of caffeine provided by the natural caffeine extract. Preferably the compositions comprise a total of 0.5 to 2.2% w/v caffeine.

In preferred embodiments, the pharmaceutical composition also comprises a pharmaceutically acceptable carrier. Preferably the composition is in the form of a solution, preferably an isotonic solution. Preferably the pharmaceutical composition comprises 1.5-2.1% or 1.5-2.0% w/v caffeine. In some embodiments, the natural caffeine extract comprises 8-30% v/v of the composition. In some embodiments, the aqueous pharmaceutical composition comprises at least one pharmaceutically acceptable excipient. In some preferred embodiments, a pharmaceutically acceptable excipient is a preservative.

In yet another aspect, there is provided a pharmaceutical composition formulated for nasal administration comprising:
natural caffeine extract;
at least one pharmaceutically acceptable excipient;
a pharmaceutically acceptable aqueous carrier; and, optionally, additional caffeine or a pharmaceutically acceptable salt, derivative, metabolite or solvate thereof.

In a yet further aspect, there is provided a pharmaceutical composition formulated for nasal administration comprising, consisting or consisting essentially of:
natural coffee extract;
at least one pharmaceutically acceptable excipient;
a pharmaceutically acceptable aqueous carrier; and, optionally, additional caffeine or a pharmaceutically acceptable salt, derivative, metabolite or solvate thereof.

In a preferred embodiment, there is provided a pharmaceutical composition comprising, consisting or consisting essentially of:
coffee extract: 8-30% v/v;
additional caffeine: 0.5-2% w/v;
potassium sorbate: 0.02-0.2% w/v;
glycerol: 0.5-2% v/v;
hypromellose: 0.01-1% w/v; and
phosphate buffered saline: 70-90% v/v.

There is also provided a method of for treating or preventing a craving for coffee, or treating or preventing caffeine withdrawal or a symptom of caffeine withdrawal in an individual comprising administering to the individual via intranasal delivery a pharmaceutical composition of the invention.

In a further aspect there is provided a pharmaceutical composition of the invention for use in the manufacture of a medicament for treating or preventing a craving for coffee, or treating or preventing caffeine withdrawal or a symptom of caffeine withdrawal.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much 15, 14, 13, 12, 11, 10, 9, 8, 7, 6, 5, 4, 3, 2 or 1% to a reference quantity, level, value, number, frequency, percentage, dimension, size, amount, weight or length.

When used herein the terms “% w/w”, “% w/v” and “% v/v” mean, respectively, weight to weight, weight to volume, and volume to volume percentages.

As used herein, the term “and/or” refers to and encompasses any and all possible combinations of one or more of the associated listed items, as well as the lack of combinations when interpreted in the alternative (or).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. Thus, the use of the term “comprising” and the like indicates that the listed integers are required or mandatory, but that other integers are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

When used herein, the term “natural caffeine extract” refers to a solvent extract containing caffeine obtained from one or more caffeine containing plants, or one or more parts of such a plant. Caffeine containing plants or parts of plants include, but are not limited to, coffee beans (green or roasted); tea leaves (white, green or back); cocoa (powder, nibs); guarana seeds; kola nuts or yerba maté leaves. Roasted coffee beans are preferred as these impart an aroma comparable to the aroma of a coffee beverage. Suitably, the extract is prepared by treating the plant matter with a suitable solvent, for example water or ethanol, using techniques well known in the art for extracting organic chemical components from plant material. It will be appreciated that such an extract is a complex mixture of chemical components, whose composition with regard to quantity and ratio of compounds is determined by various factors such as the material extracted, and the extraction conditions such as solvent system and temperature. The skilled person will appreciate that it is unlikely that any two similarly obtained extracts will be identical in composition. In preferred embodiments, the extract is an aqueous extract obtained by water extraction. If required, the extract may be concentrated by removal of at least a portion of the solvent to increase the concentration of caffeine and other chemical components. In preferred embodiments, the natural caffeine extract is coffee extract. In some embodiments more than one natural caffeine extract may be utilized; for example use of a coffee extract in combination with a cocoa extract is contemplated. In some preferred embodiments, where the natural caffeine extract is a coffee extract, the coffee extract is substantially free of any other natural caffeine extract.

When used herein the term “coffee extract” refers to a natural coffee extract obtained from coffee beans (seeds from a variety of the Coffea plant). Coffee beans may be sourced from several varieties of coffee plants such as Coffea Arabica or Coffea Canephora Robusta. Coffee extracts are readily available from commercial sources, or may be prepared by known methods.

The coffee extract is preferably prepared in a concentrated form to maximize the concentration of the chemical components, such as caffeine, other xanthines and aromatic molecules, extracted from the bean. The coffee beans may be in their raw, or green, state; however it is preferred that the coffee beans are roasted prior to extraction to impart a distinctive “roasted coffee” aroma to the nasal spray. Roasted coffee beans are generally considered to contain 6 mg to 10 mg of coffee per gram of coffee beans, depending on the variety and degree of roasting. For example, Robusta coffee beans generally comprise more caffeine than Arabica beans; and heavily roasted coffee beans are usually lower in caffeine than lightly roasted beans as prolonged roasting can remove a portion of the caffeine from the beans through sublimation.

The coffee beans may be extracted whole, however it will be appreciated that the extraction process will be more efficient if the beans are first broken down by, for example, cracking, grinding, crushing or otherwise breaking down the bean using any one of several well-known methods. Preferably the roasted coffee beans are ground using a conventional coffee mill or grinder. It will be appreciated that various techniques may be employed to obtain a coffee extract. Any suitable method of extracting the chemical constituents from the beans may be used. For example, the beans may be soaked for the desired period of time in a solvent under ambient conditions or elevated temperatures. Elevated temperatures may increase the efficiency of caffeine extraction. Extraction efficiency may also be increased by utilizing pressure, for example using conventional apparatus and techniques for producing espresso coffee from ground, roasted coffee. A suitable solvent may be, for example, water or an organic solvent such as ethanol, however water is preferred. The temperature used for the extraction will depend on the solvent used. Temperatures from 20° C. to 100° C. are considered to be typical. It will be appreciated that any organic solvent used in the extraction process should be considered safe for human use in the quantity ultimately present in the dosage of the nasal composition. Preferably the coffee extract is an aqueous extract obtained from ground, roasted coffee beans using any suitable aqueous extraction technique.

Aqueous extraction has an advantage that the extract produced will be likely to comprise a similar range and distribution of chemical components, particularly those comprising the aromatic properties, to that found in a typical coffee beverage. The temperature at which the ground coffee bean is extracted will vary according to the actual process used. In some processes, water used in the extraction is at a temperature of approximately 30-100° C., 50-100° C., 70-100° C., 90-100° C., or approximately 100° C. In some extraction processes, preferably the temperature is approximately 90-96° C. or 91-94° C. In some extraction processes, the solvent may be at ambient temperature or lower. The pressure used during extraction of the ground coffee beans will vary according to the method used. Suitable pressures include ambient pressure or, for example, 5-20 bar, depending on the extraction method. For example, an aqueous coffee extract is conveniently obtained using a conventional commercial catering or domestic espresso machine. It will be appreciated that an aqueous coffee extract may be obtained using other well-known means for preparing coffee beverages such as filter, drip filter, cafetière, percolator, briki, and the like. A coffee extract may also be prepared using a domestic or commercial coffee machine comprising a pump which prepares coffee beverages by pumping hot water under pressure through a pre-packaged single use coffee pod or coffee capsule containing ground roasted coffee beans. Typical pressures are 9-20 bar, for example about 19 bar. Such coffee machines are readily available from well-known manufacturers such as De'Longhi, Breville, Magimix, Bosch, Dualit, and the like. Alternatively, extraction of the ground roasted coffee beans may be effected using an aqueous extraction method such as those typically employed in scientific laboratories, for example using a Soxhlet extraction.

In some embodiments, the coffee extract may comprise 2 mg/mL to 10 mg/mL or 2 mg/mL to 5 mg/mL caffeine, for example 3 mg/mL to 5 mg/mL or 2 mg/mL to 4 mg/mL of caffeine. Methods of determining the amount of caffeine present in a coffee extract are well known in the art and include quantitative analysis using, for example, high-pressure liquid chromatography (HPLC) or gas chromatography (GC). Coffee extracts in the form of espresso coffee prepared using a conventional espresso machine or pod machine may be used in the preparation of a pharmaceutical composition without further processing steps. However, it is preferable to remove any insoluble particulate matter from the coffee extract using well-known techniques such as centrifugation, decanting and/or filtering. Depending on the method of extraction, and if necessary or desired, the coffee extract may be concentrated by removing water to increase the concentration of chemical components such as caffeine. Excess water is conveniently removed under reduced pressure. This may be effected by well-known methods such as vacuum distillation, or by use of commercially available equipment such as a rotary or film evaporator. After concentration, the coffee extract may be treated to remove any particulate matter, as described above. Coffee extract will generally comprise a mixture of polyphenol compounds, xanthine compounds and a variety of other small molecule organic compounds which contribute to the characteristic coffee aroma. The main xanthine compound present in coffee extract is caffeine, with other xanthine compounds such as theobromine and theophylline being present in smaller amounts. It will be appreciated that a coffee extract is a complex mixture of chemical components, and the range of components and relative amounts present will vary. The exact composition of a coffee extract will vary according to several factors such as the variety of coffee bean; the conditions under which it has been cultivated; the degree of roasting; the degree of grinding; the solvent used; and the extraction conditions including temperature, time, pressure, and equipment used. It will also be appreciated that the aroma of the coffee extract will also depend on several factors including the roasting conditions, extraction solvent, extraction conditions (time, temperature, pressure), variety of coffee beans and growing conditions.

Organic compounds that may contribute to the characteristic aroma of roasted coffee beans include small organic molecules, for example furans, pyrazines, phenols, aldehydes, ketones and sulphur compounds such as thiols. Specific compounds known to contribute to the characteristic aromas of coffee include 2-furfurylthiol, 3-methyl-2-buten-1-thiol, methane thiol, methylpropanal, 3-methylbutanal, acetaldehyde, 3-mercapto-3-methylbutylformate, (E)-β-damascenone, guaiacol, furaneol, 2-isobutyl-3-methoxypyrazine and 2-ethyl-3,5-dimethylpyrazine. It will be appreciated that one or more of these compounds would be expected to be present in a natural coffee extract. These organic compounds are relatively small, each having molecular masses of less than 1000 Da. Accordingly, they are believed to be able to penetrate easily the mucosal layers of the nasal cavity.

Caffeine (IUPAC name: 1,3,7-trimethylpurine-2,6-dione) is a bitter tasting and odourless alkaloid of the methylxanthine family. It is a central nervous system stimulant and is known to reversibly block the action of adenosine at the adenosine receptor, and thus can prevent the onset of drowsiness induced by adenosine. Caffeine is metabolized in the liver to give dimethyl xanthine compounds such as paraxanthine, theobromine and theophylline.

Caffeine is commonly found in coffee and tea beverages, as well as in cola (kola), yerba maté and guarana drinks. An average serving of espresso coffee is likely to provide 80-120 mg caffeine. A daily oral intake of 400 mg of caffeine in a healthy adult is generally considered to be safe. Intranasal caffeine can elicit a systemic therapeutic response and provide enhanced bioavailability at low dosage levels. Rapid onset of action can be expected. Caffeine is commercially available, and may be in the form of an anhydrate; a pharmaceutically acceptable salt; or a solvate, for example a hydrate. Preferably caffeine used in the preparation of nasal compositions described herein to supplement the caffeine provided by the natural caffeine extract and increase the concentration of caffeine of the nasal compositions is of a purified form having greater than 98.5% purity. British Pharmacopoeia (BP) or US Pharmacopoeia (USP) grade caffeine is preferred, and is readily available from commercial sources. In some embodiments, the caffeine used in the preparation of the compositions described herein is anhydrous caffeine. Use of other methylxanthine compounds, for example 1-methyl xanthine, 3-methyl xanthine, 7-methyl xanthine, theophylline or theobromine, is contemplated instead of, or in addition to, caffeine, although caffeine is preferred.

As used herein, the term “salts”, “derivative” and “solvate” include any pharmaceutically acceptable salt, derivative, or solvate or any other compound which, upon administration to the recipient, is capable of providing caffeine. Suitable pharmaceutically acceptable salts of caffeine include citrate, hydrochloride and sodium benzoate. Pharmaceutically acceptable solvates are known in the art, and include hydrates and alcoholates. Suitably, pharmaceutically acceptable solvates include hydrates, for example monohydrates, dihydrates and trihydrates. In some embodiments, caffeine is in the form of an anhydrate. Suitable pharmaceutically acceptable derivatives include ethers or esters. The preparation of salts, derivatives and solvates can be carried out using methods well known in the art.

The term “nil-by-mouth” when used herein refers to a fasting protocol imposed on a patient, usually prior to a medical procedure. Generally this involves the withholding of all food and fluid by mouth, but may not be so limited. It will also be appreciated that an individual may choose to avoid consuming, for example, a coffee beverage as it may be considered inconvenient or disadvantageous to do so.

The term “caffeine withdrawal” when used herein refers to a condition including one or more symptoms resulting from abstaining from coffee (or other caffeinated beverage) for a period of time. Caffeine is a central nervous system stimulant and regular use can cause a dependence. Caffeine withdrawal symptoms may occur if caffeine ingestion is stopped, and may last for several days. Symptoms associated with caffeine withdrawal may vary and will depend on the individual, the level of their usual caffeine intake and the rapidity of the decrease in caffeine intake. Caffeine withdrawal symptoms may include one or more of headache, including severe headache and hemicrania, irritability, dysphoria, lethargy, drowsiness, depression, lack of concentration, muscle pain, stiffness, cramping, insomnia, nausea, vomiting, constipation, anxiety, dizziness, heart palpitations, heart rhythm abnormalities, low blood pressure and reduced performance in mental or physical tasks (“brain fog”).

“Coffee craving” when used herein refers to a desire or a perceived need in an individual to consume a coffee beverage, or similar caffeinated beverage such as tea, cola or cocoa. The level of the desire may, in some circumstances, be a strong desire or perceived need.

The term “subject” or “individual” as used herein refers to a vertebrate subject, particularly a mammalian subject, for whom therapy or prophylaxis is desired. In particular embodiments, the subject is a human.

The terms “alleviate”, “treat”, “treating” or “treatment” as used herein cover the treatment of caffeine withdrawal and/or a symptom of caffeine withdrawal and/or coffee craving and include: inhibiting the condition, i.e., arresting its development; relieving the condition, i.e., causing regression of the condition; or relieving the symptoms resulting from the condition without addressing the underlying disease or condition.

Each embodiment described herein is to be applied mutatis mutandis to each and every embodiment unless specifically stated otherwise.

### 2. Methods of the Invention

The present invention is based on the surprising discovery that caffeine withdrawal, or one or more symptoms of caffeine withdrawal, or a craving for coffee, can be prevented or treated by intranasal administration to an individual in need thereof a composition comprising a natural caffeine extract and, optionally, additional caffeine.

It is believed that intranasal administration of a natural caffeine extract as described herein provides access to an effective and convenient treatment of caffeine withdrawal or coffee craving. Caffeine is known to be capable of being absorbed and transported to the brain though the nasal epithelium. Without being bound by theory or mode of action, it is believed that, following nasal administration of a pharmaceutical composition comprising a natural caffeine extract in accordance with the present invention, the caffeine is then transported directly from the nasal cavity, particularly the olfactory region at the roof of the nasal cavity, to the cerebrospinal fluid and/or brain tissue where it can produce the desired pharmacological effect. This mechanism of reaching the cerebrospinal fluid or brain tissue may involve caffeine crossing the epithelial layer by one or more of several mechanisms known in the art, such as via an olfactory pathway; through the trigeminal nerve; or through vascular pathways in the respiratory of olfactory regions. It will be understood that natural caffeine extracts as referred to herein may comprise additional xanthine compounds which may also have pharmacological effect. It is believed that these compounds will also be capable of being transported from the nasal cavity to the cerebrospinal fluid or the brain by a similar mechanism.

Administration of natural caffeine extract by an intranasal route provides several advantages. For example, the intranasal route provides access to a large surface area of the epithelium to maximize caffeine absorption. Caffeine absorption is believed to be rapid due to the existence of a porous, highly vascularized epithelium and a porous basement membrane, thus promotion of rapid onset of physiological action is expected. Furthermore, the caffeine is believed to be absorbed directly into the systemic circulation or into the central nervous system (CNS). This has an additional advantage of avoiding conventional “first pass” metabolism of orally administered caffeine in the liver and gastrointestinal tract. Importantly, it is also believed that intranasal delivery is a useful approach for caffeine administration as this provides a mechanism for being able to bypass the blood-brain barrier. Thus, intranasal administration may be more efficient than an enteric route, and is therefore believed to contribute to a more rapid onset of relief of caffeine withdrawal.

Intranasal administration is amenable to self-medication, and this is believed to contribute to a reduced risk of over-dosage. Moreover, as the onset of drug action following intranasal administration is rapid, this also contributes to reducing risk of caffeine overdose as the time lag between caffeine administration and onset of action is reduced.

Caffeine absorbed though the nasal epithelium avoids the digestive pathway. Thus, intranasal administration of caffeine can also allow the individual to maintain a nil-by-mouth status. This is particularly convenient for individuals required to fast during preparation for a medical procedure.

In preferred embodiments, the natural caffeine extract is a coffee extract. The coffee aroma provided by the coffee extract in the composition is believed to provide a pleasing and satisfying feeling reminiscent of drinking a cup of coffee. In addition to the effect of the caffeine on the central nervous system, it is believed that the familiar and attractive coffee aroma provided by the coffee extract may assist in relieving stress and anxiety in patients awaiting a medical procedure such as surgery or blood withdrawal. This is supported by observations on the benefits of coffee aroma demonstrated in animal proteomic studies [see H.-S. Seo, M. Hirano, J. Shibato, I.-K. Hwang, Y. Masuo: “Effects of coffee bean aroma on the rat brain stressed by sleep deprivation: a selected transcript- and 2D gel-based proteomic analysis”, Journal of Agricultural and Food Chemistry, 56 (2008) 4665-4673]. Furthermore, purified caffeine is an odourless and bitter tasting chemical, therefore intranasal administration of a caffeine composition may be considered by some to be unpleasant, and is unlikely to provide a level of satisfaction associated with consuming a cup of coffee or other similar caffeinated drink. The use of natural caffeine extract in the nasal composition provides a more acceptable and pleasant experience. In particular, the preferred natural coffee extract provides the aromas of ground roasted coffee. It is believed that this aroma can also assist in satisfying coffee cravings which may be particularly exacerbated in an individual awaiting a medical procedure due to the presence of coffee bars and vending machines which may produce coffee aromas in the vicinity of waiting rooms, hospitals, surgeries and clinics.

Accordingly, there is provided a method of treating or preventing a craving for coffee, caffeine withdrawal or a symptom of caffeine withdrawal in an individual comprising administering to the individual via intranasal delivery an aqueous pharmaceutical composition comprising a natural caffeine extract and at least one pharmaceutically acceptable excipient.

The method of treating or preventing a craving for coffee, caffeine withdrawal or a symptom of caffeine withdrawal can be employed in any situation where caffeine administration is desired or beneficial, and may be useful to any individual where administration of caffeine is not contraindicated. Particular individuals include, but are not limited to, machine operators; vehicle operators, such as long-distance drivers; nightshift workers; students; athletes; military personnel; weight watchers; and the like. In some embodiments, the methods of the invention are used to treat, prevent or alleviate a craving for coffee in an individual. In some embodiments, the methods may be used to treat or prevent a symptom of caffeine withdrawal.

Caffeine can be absorbed and transported though the nasal epithelium, thus avoiding the digestive pathway. Thus, the intranasal delivery method permits the individual to maintain a nil-by-mouth status. Accordingly, there is also provided a method of alleviating caffeine withdrawal or a symptom of caffeine withdrawal or craving for coffee whilst maintaining a nil-by-mouth status comprising administering to the individual via intranasal delivery an aqueous pharmaceutical composition comprising a natural caffeine extract and at least one pharmaceutically acceptable excipient. In some embodiments, there is provided a method of alleviating caffeine withdrawal or a symptom of caffeine withdrawal or craving for coffee whilst maintaining a nil-by-mouth status comprising administering to the individual via intranasal delivery an aqueous pharmaceutical composition comprising, consisting or consisting essentially of coffee extract and at least one pharmaceutically acceptable excipient.

The natural caffeine extract may be obtained from caffeine containing plants, or parts thereof, selected from, for example tea leaves, coffee beans, yerba maté leaves, kola nuts, guarana seeds or cocoa powder or nibs. In some preferred embodiments the natural caffeine extract is a coffee extract.

Accordingly, in a preferred aspect, there is provided a method of treating or preventing a craving for coffee in an individual, or treating or preventing caffeine withdrawal or a symptom of caffeine withdrawal in an individual, comprising administering to the individual via intranasal delivery an aqueous pharmaceutical composition comprising, consisting or consisting essentially of a coffee extract and at least one pharmaceutically acceptable excipient. There is also provided a method of alleviating caffeine withdrawal or a symptom of caffeine withdrawal or craving for coffee whilst maintaining a nil-by-mouth status comprising administering to the individual via intranasal delivery an aqueous pharmaceutical composition comprising, consisting or consisting essentially of a coffee extract and at least one pharmaceutically acceptable excipient.

In another aspect, there is provided an aqueous pharmaceutical composition comprising, consisting or consisting essentially of a coffee extract and at least one pharmaceutically acceptable excipient for use in treating or preventing a craving for coffee, or treating or preventing caffeine withdrawal or a symptom of caffeine withdrawal, wherein the composition is adapted for intranasal delivery.

In a yet further aspect, there is provided a use of an aqueous pharmaceutical composition comprising, consisting or consisting essentially of a coffee extract and at least one pharmaceutically acceptable excipient, in the manufacture of a medicament for treating or preventing a craving for coffee, or treating or preventing caffeine withdrawal or a symptom of caffeine withdrawal, wherein the composition is adapted for intranasal delivery.

It will be appreciated that the amount of caffeine present in a natural caffeine extract will depend on many factors such as the species and variety of the plant, the growth conditions and the extraction conditions used to prepare the extract. Coffee extract, when prepared according to the methods described herein, typically contains plant derived caffeine in an amount of from about 2 mg/mL to about 10 mg/mL, or 0.002 g/mL to 0.01 g/mL. Caffeine has a maximum solubility in aqueous solutions of from 0.018 to about 0.022 g/mL, or approximately 0.02 g/mL. Therefore, in order to maximize the amount of caffeine present in the coffee extract or in the nasal composition, additional caffeine may be added. For example, additional caffeine may be added to an aqueous nasal composition comprising coffee extract, or to a coffee extract, as described herein to increase the total amount of caffeine present in the aqueous pharmaceutical composition to a maximum of approximately 2.0, 2.1 or 2.2% w/v caffeine; for example approximately 0.5-2% w/v, 0.5-2.2% w/v, 1.0-2.2% w/v or 1.5-2.2% w/v.

The methods or uses described herein may be employed to prevent or treat one or more symptoms associated with caffeine withdrawal selected from headache, severe headache, hemicrania, irritability, dysphoria, lethargy, drowsiness, depression, lack of concentration, muscle pain, joint pain, stiffness, cramping, insomnia, stomach pain, abdominal pain, nausea, vomiting, constipation, anxiety, dizziness, heart palpitations, heart rhythm abnormalities, low blood pressure, and reduced mental or physical performance.

Although not limited to such, the methods or uses herein may be used to alleviate coffee craving, caffeine withdrawal or symptoms of caffeine withdrawal whilst retaining a nil-by-mouth status in an individual. Therefore the methods may be used in fasting individuals, such as those preparing for a medical procedure such as, but not limited to anaesthesia; sedation; surgery; colonoscopy; endoscopy; or gastroscopy. The methods may also be used in individuals required to fast prior to drawing of blood for testing for blood cholesterol and lipid concentration, blood glucose concentration, iron levels, vitamin B12 levels, metabolic information, or kidney or liver function. Methods described herein may also have application to individuals prior to radiographic investigations such as positron emission tomography (PET) scans or those investigations where food residue in the gastrointestinal tract may impact negatively on the quality of the imaging of some organs such as the liver, gallbladder, spleen or pancreas.

In the methods or uses described herein, the aqueous composition is delivered intranasally. This allows the caffeine to be transferred through the nasal epithelium and directly into the systemic blood circulation, whilst avoiding the digestive system. Suitably the methods of the invention involve self-administration of the caffeine extract composition. Spray administration is a preferred method for delivering or administering the nasal compositions of the invention, with pump sprays being particularly preferred. Spray administration deliver the composition as a mist which maximizes the effect of the coffee aroma. Suitable containers or devices of appropriate dimensions and shape for nasal spray administration or delivery are well known in the art, and include spray containers such as pump sprays. Suitable spray applicators for nasal delivery of aqueous compositions are commercially available and may include crimp-on or snap-on nasal pumps, for example those available from Bona Pharma, People's Republic of China (www.bona-cn.com). Typically, a bottle of the spray applicator has a volume of 10 mL to 20 mL. In some embodiments the spray applicator has a volume of approximately 15 mL, making it a convenient size for transportation and use. In one embodiment, a nasal spray applicator is a metered dose device adapted to provide a measured dose per actuation. It is desirable that the volume of composition delivered by intranasal delivery is minimized for comfort and convenience to the individual. This also prevents the possibility of significant volumes of caffeine composition being ingested if the individual is fasting. Suitably the volume of composition delivered per actuation is from 0.05-0.2 mL, for example 0.05-0.15 mL or approximately 0.1 mL. In some embodiments, the amount of caffeine delivered per actuation is approximately 1-4 mg, for example approximately 2 mg. In some embodiments, a typical dose delivers 2 to 10 mg of caffeine, for example 2 to 5 mg. In one embodiment, approximately 4 mg of caffeine is administered, with approximately 2 mg being delivered to each nostril.

Aqueous pharmaceutical compositions comprising a natural caffeine extract, for example a coffee extract, and, optionally, additional caffeine or a pharmaceutically acceptable salt, solvate or derivative thereof are preferably in the form of a solution. It will be appreciated that a solution adapted for nasal delivery will preferably be non-toxic, non-irritant and/or isotonic. In preferred embodiments, the composition is preferably of a pH compatible with nasal administration, for example pH 5-8. Preferably the composition has a pH of 5-7.5, for example approximately pH 7.4. The pharmaceutical composition is formulated in a pharmaceutically acceptable aqueous carrier, for example purified water or saline. In a preferred embodiment, the aqueous carrier is buffered saline solution, for example phosphate buffered saline.

Aqueous pharmaceutical compositions comprising a natural caffeine extract and, optionally, additional caffeine are preferably in the form of an aqueous solution. The maximum solubility of caffeine in water under ambient conditions is generally recognized to be approximately 2 g/100 mL (2% w/v), accordingly it will be understood that the maximum concentration of caffeine in an aqueous solution will be substantially 2% w/v. It will be appreciated that the solubility of caffeine in a solvent will depend on factors such as pH, temperature, type of solvent and amount and type of other solutes present. An aqueous composition comprising caffeine and natural caffeine extract for nasal administration will suitably comprise 0.5-2% w/v of anhydrous caffeine and 8-30% v/v or 10-30% v/v of caffeine extract, for example 10-30% v/v coffee extract. Preferably the aqueous composition will comprise about 1-2% w/v caffeine, 1.5-2% w/v caffeine, 1.8-2% w/v caffeine or approximately 2% w/v caffeine, wherein the caffeine is derived from anhydrous caffeine. It will be appreciated that the coffee extract used in the preparation of the composition will also contain caffeine, which will contribute to the overall percentage of caffeine in the composition. Typically the coffee extract will comprise approximately 2 mg/mL to 10 mg/mL or 2 mg/mL to 5 mg/mL caffeine, and typically may contribute 0.05-0.1% w/v or 0.05-0.2% w/v caffeine to the composition. In some embodiments, the aqueous composition comprises 0.5-2.2% w/v caffeine, for example 0.5-2.1% w/v, 1.8-2.1% w/v or 1.8-2.0% w/v total caffeine.

The pharmaceutical composition comprises at least one pharmaceutical excipient selected from, for example, preservatives, humectants, buffering agents, flavouring agents and tonicity agents. In one preferred embodiment, an excipient is a preservative.

It will be appreciated that the compositions may, in addition to caffeine, further comprise one or more therapeutically active agents. It will be appreciated that the active agents are preferably bioavailable when administered by an intranasal route. Additional therapeutically active ingredients may include analgesics, anti-inflammatory or antipyretic agents which are bioavailable by intranasal delivery. In some embodiments, additional therapeutically active ingredients may include theobromine, methyl xanthine, paraxanthine or aminophylline.

Preferably, when the composition is administered by a spray applicator, the applicator is adapted to provide a pre-determined volume of the composition per actuation, for example 0.05 mL to 0.2 mL per actuation, especially approximately 0.1 mL per actuation. Typically 0.1 mL of composition will contain approximately 0.5-2.1 mg of caffeine.

The dosage and frequency of administration of the composition will depend on the requirements of the individual. In some embodiments, the composition may be self-administered when desired or required provided that this is not contraindicated due to the presence of an existing medical condition or by the need for administration of certain medication. Administration of 2-8 mg, or 2-6 mg, of caffeine per dose is contemplated. For example, administration of approximately 4 mg of caffeine per dose is considered to be suitable. In some embodiments, administration of approximately 2 mg caffeine per nostril, is contemplated. It is believed that self administration, together with rapid onset of pharmacological action, may contribute to reducing the likelihood of overdosing with caffeine.

It has been observed that a caffeinated nasal spray composition as described herein is pleasant, convenient and easy to administer and has been used to good effect. Nasal administration may provide an approximate dose equivalent to a sip of strong espresso coffee, however the onset of a caffeine effect may be almost instantaneous. The nasal spray may be re-administered as needed to titrate to optimum desired effect. The aroma may be described as that of freshly ground coffee beans and translates from the aromatic characteristics of the original coffee beans used for the extraction process. The pleasant aroma has been reported to linger for 30 minutes or more from administration of a single nasal spray.

### 3. Compositions of the Invention

The inventors have discovered that intranasal administration of an aqueous solution comprising a natural caffeine extract, such as coffee extract, and, optionally, additional caffeine can alleviate the symptoms of caffeine withdrawal or coffee craving in an individual. In particular, intranasal administration can maintain a nil-by-mouth status in the individual. Furthermore, it has also been discovered that an aqueous composition comprising a natural coffee extract provides a coffee aroma on administration and can provide a satisfying and authentic coffee experience at times where drinking coffee is not convenient or not possible. It is also believed that the coffee aroma can assist in relieving stress and anxiety, which may be particularly useful for many patients awaiting a medical procedure.

Accordingly, in a further aspect, the present invention further provides a pharmaceutical composition comprising:
at least one natural caffeine extract;
at least one pharmaceutically acceptable excipient;
a pharmaceutically acceptable aqueous carrier; and, optionally caffeine or a pharmaceutically acceptable salt, solvate, derivative of metabolite thereof.

In a preferred embodiment, the pharmaceutical composition comprises:
at least one natural caffeine extract;
at least one pharmaceutically acceptable excipient;
a pharmaceutically acceptable aqueous carrier; and, optionally caffeine or a pharmaceutically acceptable salt, solvate, derivative of metabolite thereof;
wherein the composition comprises total caffeine in an amount of 0.5-2.2% w/v.

The composition is preferably formulated as a composition adapted for, or suitable for, nasal administration. Preferably the pharmaceutical composition is an aqueous solution, for example an isotonic solution. It will be appreciated that the carrier(s) and excipient(s) must be “acceptable” in the sense of being compatible with the other ingredients of the composition and not deleterious to the recipient thereof. In some embodiments the aqueous carrier is water. It will also be understood that a composition for nasal administration is preferably of a physiologically acceptable pH, for example pH 5-7.5. Preferably the aqueous carrier is buffered to pH 5-7.5, for example pH 7.4. In some embodiments, the aqueous carrier is saline solution, such as buffered saline solution, preferably phosphate buffered saline solution. Preferably, the osmolality and ion concentrations of the aqueous carrier are similar to those of the human body (isotonic) and are non-toxic to most cells. Preferably the aqueous carrier is non-irritant. In some embodiments, the aqueous carrier comprises 0.1-0.2 M sodium chloride, for example 0.15-0.16 M sodium chloride or approximately 0.154 M sodium chloride. In some embodiments, the aqueous composition comprises 0.138 M NaCl and 0.0027 M KCl. In some embodiments, the aqueous carrier is 0.01M phosphate buffered saline (PBS; pH 7.4 at 25° C.), containing 0.138 M NaCl and 0.0027 M KCl.

Natural caffeine extracts are preferably selected from coffee extract, or extracts of tea, cocoa, kola, guarana or yerba maté. In a preferred embodiment, the natural caffeine extract is coffee extract. In one embodiment, the natural caffeine extract consists of coffee extract. Preferably the coffee extract is derived from roasted coffee beans.

Accordingly, in a further aspect, the present invention further provides a pharmaceutical composition comprising, consisting or consisting essentially of:
coffee extract;
at least one pharmaceutically acceptable excipient;
a pharmaceutically acceptable aqueous carrier; and, optionally caffeine or a pharmaceutically acceptable salt, solvate, derivative of metabolite thereof.

In one embodiment, there is provided a pharmaceutical composition comprising, consisting or consisting essentially of:
coffee extract;
at least one pharmaceutically acceptable excipient;
a pharmaceutically acceptable aqueous carrier; and, optionally caffeine or a pharmaceutically acceptable salt, solvate, derivative of metabolite thereof;
wherein the composition comprises total caffeine in an amount of 0.5-2.2% w/v.

Preferably the coffee extract is a natural coffee extract derived by aqueous extraction of roasted, ground coffee beans. It will be appreciated that the coffee extract will comprise caffeine and other xanthine compounds such as theobromine and theophylline in addition to polyphenolic compounds and varying amounts of a variety of small organic molecules which may contribute to the characteristic aroma of roasted coffee beans. In some embodiments, the coffee extract comprises caffeine in an amount of 2 mg/mL to 10 mg/mL or 2 mg/mL to 5 mg/mL, for example 2.5 mg/mL to 4.5 mg/mL; 3.0 mg/mL to 4.0 mg/mL; or 3.0 mg/mL to 3.5 mg/mL. In addition to caffeine, it will be understood that other xanthine compounds, for example theobromine or theophylline, are also likely to be present in the coffee extract. These will generally be present in smaller amounts than caffeine. In some embodiments, a composition of the invention comprises coffee extract in an amount of 5-50% v/v, for example 10-40% v/v, 8-30% v/v, 10-35% v/v, 15-30% v/v 15-25% v/v or 10-30% v/v.

Coffee extracts prepared in accordance with the examples of the present invention have not been found to be particularly susceptible to forming a sediment, either in the extract or in the resulting intranasal composition. However, on some instances, a coffee extract may form a sediment due to the presence of certain carbohydrate components in the extract. If deemed necessary or advantageous, one or more additives, such as a carbohydrate-hydrolysing enzyme may be added to the coffee extract to reduce or avoid the possibility of sedimentation due to the presence of carbohydrates. Examples of such enzymes include mannanases; see, for example U.S. Pat. Nos. 2,282,139 and 2,801,920.

Carbohydrate-hydrolyzing enzymes are known in the art, and include endo-1,4-β-mannanase (Megazyme, Bray, Ireland); Gamanase™ (Novozymes, Bagsvaerd, Denmark), which contains a mixture of endo-1,4-β-mannanase, exo-1,4-β-mannosidase, and α-galactosidase; and Rohapect™ B1L (AB Enzymes, Germany), which contains pectinases. If such an enzyme is desired or required, it is preferably added to the coffee extract after the extraction step and prior to a final filtration step before incorporating into a caffeine composition as described herein.

Although the coffee extract, and therefore the pharmaceutical compositions prepared from the coffee extract, comprise caffeine; in preferred embodiments it is desirable to include additional caffeine in the pharmaceutical composition to raise the total caffeine content. In preferred embodiments, additional caffeine is included in an amount to bring the total caffeine content of the composition up to approximately 2.2% w/v, or 2.1% w/v or about 2% w/v. Caffeine used in the preparation of the compositions as additional caffeine is preferably of high purity, and of food grade or pharmaceutical grade or quality. Preferably the additional caffeine is of pharmaceutical grade, with a purity of greater than 98%, for example greater than 98.5% or greater than 99% pure. The additional caffeine may be in the form of an anhydrate or as a pharmaceutically acceptable salt or solvate. It may also be used in the form of a caffeine derivative or metabolite. In one embodiment, the additional caffeine is commercially available pharmaceutical grade anhydrous caffeine. In some embodiments, the composition is prepared from 0.5-2.0% w/v anhydrous caffeine in addition to the coffee extract. In some embodiments, the composition is prepared from 0.5-2% w/v, for example 0.5-2% w/v, 0.8-2% w/v, 1-2% w/v, 1.5-2% w/v or 1.8-2% w/v anhydrous caffeine in addition to the coffee extract.

In addition to the natural caffeine extract, additional caffeine (where used) and aqueous carrier, the compositions of the invention also comprise one or more additional ingredients selected from buffering agents, stabilizers, tonicity agents, humectants, thickening agents; flavouring agents; and preservatives. In some embodiments, the composition may also comprise a component, such as one or more enzymes, to reduce or negate any likelihood of sedimentation.

The pharmaceutical compositions of the present invention, or the compositions used in the methods of the present invention, may be formulated and administered using methods well known in the art. Techniques for formulation and administration may be found in, for example, Remington: The Science and Practice of Pharmacy, Loyd V. Allen, Jr (Ed), The Pharmaceutical Press, London, 22^{nd} Edition, September 2012.

It will be appreciated that it may be useful to incorporate one or more pharmaceutically acceptable excipients in the composition. Excipients for aqueous compositions include, but are not limited to, buffers, stabilizers, tonicity agents, humectants, antioxidants, thickening agents, viscosity modifiers, rheology modifiers, flavouring agents and preservatives. Suitable excipients are well known in the art and are readily available from commercial sources. Preferably, the excipients are of pharmaceutical grade, for example USP or BP grade. Pharmaceutical excipients are described in, for example, Handbook of Pharmaceutical Excipients, Paul J. Sheskey et al., The Pharmaceutical Press, London, Eighth Edition, August 2017. It will be appreciated that determination of whether a particular class of excipient is required, and selection of an appropriate excipient will be well within the skill and knowledge of a person of ordinary skill. It will also be recognized that an excipient must be chemically inert with respect to the other components in the composition. The concentration of any particular excipient will vary in accordance with its identity and the skilled person would readily be able to select suitable excipients and determine the amount necessary without undue burden or inventive input.

For example, excipients may include one or more buffering agents to maintain the pH of the composition at a physiologically acceptable pH. Suitable buffering agents are well known in the art, and include phosphate buffer, such as sodium phosphate, for example, a mixture of monosodium phosphate and disodium phosphate. Other buffering agents known in the art include acetate, phthalate or borate buffering agents.

One or more tonicity agents may be added to render the composition isotonic with the nasal tissue to reduce irritation due to osmotic shock during administration. Tonicity agents are well known to the skilled person, and include dextrose, glycerol, propylene glycol, mannitol, boric acid, sodium tartrate, potassium chloride, and sodium chloride. In some embodiments, the tonicity agent may be potassium chloride and/or sodium chloride. Preferably, the one or more tonicity agents are present at a concentration of 0.1-0.3 M.

Humectants are useful in assisting topical delivery, and one or more humectants may be included in the composition to increase the solubility of the caffeine, or increase the ability of the caffeine to penetrate skin. A humectant may also inhibit drying of the nasal membrane and prevent irritation. Suitable humectants are well known to the skilled person, and include sorbitol, propylene glycol or glycerol. In one embodiment, a suitable humectant is glycerol. In some embodiments, a humectant is present in an amount of 0.5-1.5% v/v.

The skilled person will recognize that a composition of the invention may be susceptible to microbial contamination, accordingly a preservative may be incorporated into the composition to reduce or avoid its degradation or alteration. Suitable preservatives are well known in the art, and are readily available from commercial sources. Suitable preservatives include methyl paraben, ethyl paraben, propyl paraben, sodium benzoate, benzalkonium chloride, sodium propionate and potassium sorbate. In a particular embodiment, a preservative is potassium sorbate. Typically a preservative is present in an amount of 0.02-1% w/v, for example 0.02-0.5% w/v or 0.02-0.15% w/v. In some embodiments, preservative is present in an amount of 0.02-0.25% w/v or 0.05-0.5% w/v, for example 0.05-1.5% w/v, 0.08-0.12% w/v, or about 0.1% w/v.

The skilled person will also appreciate that thickening agents may be incorporated into the composition to adjust the viscous properties to the requirements of nasal delivery. Suitable thickening agents include derivatives of cellulose, such as hydroxypropyl methyl cellulose, carboxymethyl cellulose; natural gums, such as sodium alginate, xanthan, agar or carrageenan; pectins; and gelatin. In a preferred embodiment, a thickening agent is hydroxypropyl methyl cellulose, also known by the international non-proprietary name (INN) “hypromellose”. If present, the amount of thickening agent in a composition of the invention will depend on the desired consistency. Typically a thickening agent may be present in an amount of 0.01-1% w/v.

If desired, additional flavouring agents may be used to enhance the aroma of the natural caffeine extract. For example, the roasted coffee aroma provided by a coffee extract may be enhanced by additional flavouring agents such as natural cocoa extract, natural vanilla extract or caramel flavouring.

If desired, the compositions of the invention may, in addition to caffeine, further comprise one or more physiologically active agents. Preferably, any additional physiologically active agents may be formulated as an intranasal formulation and have bioavailability when administered by an intranasal route. Additional therapeutically active ingredients may include methyl xanthine compounds such as theobromine, aminophylline or theophylline. Other therapeutically active ingredients include analgesics, anti-inflammatory or antipyretic agents.

In a further embodiment, the present invention further provides a pharmaceutical composition comprising:
natural caffeine extract;
caffeine or a pharmaceutically acceptable salt, derivative, metabolite or solvate thereof;
one or more excipients selected from buffering agents, humectants, preservatives and tonicity agents; and
an aqueous pharmaceutically acceptable carrier.

In another embodiment, there is provided a pharmaceutical composition comprising, consisting, or consisting essentially of:
coffee extract;
one or more excipients selected from buffering agents, humectants, preservatives and tonicity agents; and
an aqueous pharmaceutically acceptable carrier.

In a preferred embodiment, there is provided a pharmaceutical composition comprising, consisting, or consisting essentially of:
coffee extract;
additional caffeine or a pharmaceutically acceptable salt, derivative, metabolite or solvate thereof;
one or more excipients selected from buffering agents, humectants, preservatives and tonicity agents; and
an aqueous pharmaceutically acceptable carrier.

In some embodiments, the one or more excipients are selected from buffering agents, humectants, thickening agents, preservatives and tonicity agents.

In another embodiment, the pharmaceutical composition comprises, consists of, or consists essentially of:
coffee extract;
additional caffeine;
a pharmaceutically acceptable preservative; and
a pharmaceutically acceptable aqueous carrier.

In a particular embodiment, the pharmaceutical composition comprises, consists of, or consists essentially of:
coffee extract;
additional caffeine;
one or more tonicity agents;
one or more humectants;
one or more preservatives;
one or more buffering agents; and
an aqueous carrier.

In some embodiments, the pharmaceutical composition additionally comprises one or more thickening agents.

Preferably the coffee extract is present in the composition in an amount of 10-30% v/v, preferably 15-30% v/v, more preferably 15-25% v/v, 18-25% v/v or 18-22% v/v.

Preferably the total caffeine present in the composition is in an amount of 1-2.2% w/v, preferably 1.2-2% w/v, more preferably 1.5-2% w/v or 1.8-2% w/v, for example approximately 2% w/v.

In some embodiments, the composition is prepared from, or comprises, consists or consists essentially of:
coffee extract 15-30% v/v;
additional caffeine 1.0-2.2% w/v;
glycerol 0.8-1.5% v/v;
potassium sorbate 0.02-0.2% w/v; and
phosphate buffered saline to 100%.

In some embodiments, the composition also comprises hypromellose 0.01-1% w/v.

In one embodiment, the composition is prepared from, or comprises, consists, or consists essentially of:
coffee extract 18-22% v/v;
additional caffeine 1.8-2.2% w/v;
glycerol 0.9-1.1% v/v;
hypromellose 0.05-0.2% w/v;
potassium sorbate 0.08-0.2% w/v; and
phosphate buffered saline to 100%.

In a preferred embodiment, the composition is prepared from, or comprises, consists or consists essentially of:
coffee extract about 20% v/v;
additional caffeine about 2% w/v;
glycerol about 1% v/v;
hypromellose about 0.1% v/v;
potassium sorbate about 0.1% w/v; and
phosphate buffered saline about 79% v/v

In some embodiments, the coffee extract comprises caffeine in an amount of 0.05-0.2% w/v, for example 0.05-0.1% w/v.

There is also provided a method of treating or preventing caffeine withdrawal or a symptom of caffeine withdrawal and/or coffee craving in an individual comprising administering to the individual a pharmaceutical composition of the invention.

In a further aspect there is provided a pharmaceutical composition according to the invention for use in treatment or prevention of caffeine withdrawal and/or coffee craving. There is also provided a pharmaceutical composition according to the invention for use in therapy. The pharmaceutical composition of the invention may also be used in the manufacture of a medicament for treatment or prevention of caffeine withdrawal or a symptom of caffeine withdrawal and/or coffee craving.

Although other modes of administration may be contemplated, the compositions of the invention are primarily intended for nasal administration preferably by spray delivery.

Coffee extracts for use in preparation of compositions described herein are prepared by aqueous extraction of roasted, ground coffee beans, such as those conventionally used in the preparation of a coffee beverage. However it will be appreciated that coffee may be extracted from coffee beans using an organic solvent, for example, ethanol. However, aqueous extraction is preferred as this is likely to extract a similar range and distribution of organic compounds as that extracted during preparation of a coffee beverage from coffee beans, thus providing a similar aroma. An extract may be obtained using any suitable means known for extraction of coffee beans; however use of a conventional espresso machine is convenient, however coffee beans may be extracted using other typical means of preparing coffee beverages, such as coffee filter or percolator. Alternatively, the coffee beans may be extracted using laboratory methods typically used to prepare extracts from natural materials. For example, an aqueous extractions method such as a Soxhlet extraction, are generally considered to be convenient. In some embodiments, the coffee extract may contain approximately 2.5-5 mg/mL of caffeine. The concentration of caffeine (or any other component) in the coffee extract may be determined by means known to the person skilled in the art, and include high-performance liquid chromatography (HPLC) or gas chromatography (GC) analysis. If necessary or desired, the volume of the coffee extract obtained may be reduced, and the concentration of caffeine and other xanthines, polyphenols and other organic components increased, by removal of a portion of the water by distillation to obtain a more concentrated extract.

By way of example, a coffee extract may be prepared by Soxhlet extraction using the following method:
grinding roasted coffee beans to a medium to extra fine grade;
placing a known weight of ground roasted coffee beans in to an extraction thimble and enclosing the material within the thimble by using a second larger thimble as a capping lid;
filling a round-bottomed glass flask (still pot) to half volume with purified water, and placing the flask in a heating mantle or other heat source;
placing the thimble containing ground roasted coffee beans into the extraction chamber of a Soxhlet extractor;
fitting the Soxhlet extractor to the still pot, and attaching a water-cooled condenser at the top of the extractor;
heating the still pot to boil the water and maintaining the boiling at reflux while the extraction process completes approximately 50 extraction cycles;
allowing the extract to cool down in the still pot and then filtering it through paper filter into another round-bottomed glass flask;
concentrating the extract in rotary evaporator under vacuum to a volume approximately 10 times smaller than the initial volume; and centrifuging the concentrate and discarding the residual solid matter.

As an alternative, the coffee extract may be prepared using a conventional commercially available domestic or catering espresso machine comprising the following method:
grinding the roasted coffee beans to a medium to extra fine grade;
placing a calibrated dose of ground roasted coffee beans in the basket of the portafilter of an espresso coffee machine; tamping and trimming the dose in the basket; and inserting the portafilter into the group head of the machine; extracting the coffee in the espresso machine according to its specifications for pressurized brewing; and
centrifuging the concentrate and discarding the residual solid matter.

Further sources of natural coffee extract include coffee capsules or coffee pods intended for use in a machine to produce a single serve of coffee beverage. The coffee pods or capsules are readily available from a variety of manufacturers and generally comprise ground coffee sealed in a single use container of plastic and/or aluminium. Well known examples include Nespresso™ coffee pods available from Nestlé Nespresso SA, Lausanne, Switzerland. These pods may be used in a Nespresso™ machine manufactured by De'Longhi SPA, Italy, or Breville Group Ltd, Australia, to produce a natural coffee extract.

Commercially produced coffee extracts may also be used in the compositions of the invention. Such extracts are readily available from commercial sources and include, for example, Arabica Cold Brew Coffee Extract available from S&D Coffee Inc., Concord, USA.

Natural caffeine extracts, other than those produced from coffee beans, may be prepared from caffeine containing plants, or parts of plants. Suitable commonly used plant materials include, but are not limited to those used to prepare caffeinated beverages, for example:
Tea (white, green or fermented (black) leaves of Camellia sinensis);
Yerba maté (leaves from Ilex paraguariensis);
Kola (nuts from Cola acuminate or Cola nitida);
Cocoa (powder, nibs from seeds of Theobroma cacao);
Guarana (seeds from Paullinia cupana).

Methods for extracting caffeine and other organic molecules from plant matter are well known in the art, and include extraction by soaking the plant material in a suitable solvent such as ethanol or water. It will be appreciated that increased temperature and/or pressure will generally increase the total amount of chemical components extracted from the plant material over a given time. Finely dividing the plant material prior to extraction will also increase the efficiency of the extraction process. The composition of the natural caffeine extract, including the amount of caffeine present, may be determined by methods well known to the skilled person and those described herein. If desired, the natural caffeine extract may be concentrated by removal of excess solvent.

In order that the invention may be readily understood and put into practical effect, particular preferred embodiments will now be described by way of the following non-limiting examples.

### EXAMPLES

### Materials and Methods

Methods of preparing natural caffeine extracts, including coffee extracts, and aqueous pharmaceutical compositions for nasal administration comprising the extracts are described in the Examples below. Sources of raw materials and equipment used in the preparation of the compositions are also described.

### Example 1

### Extraction Using Soxhlet Apparatus

Roasted coffee beans (200 g, medium/dark roast espresso degree) of Coffea Arabica species (Arabica), variety Columbia Supremo Bachue, supplied by The Coffee Roaster Pty Ltd, West End, Queensland, Australia, were grinded in a coffee grinder machine (Coffee & Spice™, Breville Pty Ltd, Botany, New South Wales, Australia) for 20 seconds. An amount of 8 g of ground coffee beans was weighed with an analytical balance and placed in an extraction thimble which was then capped using a second larger thimble. A 500-mL round-bottomed glass flask was filled with 250 mL deionized water, and placed in a heating mantle. The thimble containing ground roasted coffee beans was placed into the 100-mL chamber of a Soxhlet extractor and, after fitting the extractor to the round-bottom flask and placing a water-cooled condenser at the top, the flask was heated to bring water to boiling. Boiling at reflux was maintained for about 6 hours to achieve about 50 extraction cycles (about 7 minutes per cycle). The extract was cooled down in the flask, and filtered through a paper filter (No. 2, Advantec, Toyo Roshi Kaisha Ltd., Japan) into another round-bottom flask. The flask was then attached to a rotary evaporator (Rotavapor R-215, Büchi Labortechnik AG, Flawil, Switzerland), and the liquid was concentrated under vacuum (70-75 mbar) at a temperature in the water bath maintained at 50° C., to a volume of 20-25 mL, which is approximately 10 times smaller than the initial volume. The extract was centrifuged in an Eppendorf MiniSpin® plus centrifuge (Eppendorf AG, Hamburg, Germany) for 10 minutes at 14,500 rpm, and the residual solid matter was discarded.

The skilled person will appreciate that this method of extraction is equally applicable to preparation of caffeine extracts from other plant materials such as yerba maté leaves, tea leaves, kola nuts, cocoa, guarana seeds, and the like. Preferred solvents include water, ethanol, or a mixture thereof.

### Example 2

### Coffee Extraction Using an Espresso Coffee Machine

The espresso coffee machine used was a Barista Express™ (Breville Pty Ltd, Botany, New South Wales, Australia), which has an integrated grinder. It will be appreciated that any similar domestic or commercial espresso coffee machine may be used. The roasted coffee beans (as used in Example 1) were placed in the bean hopper of the espresso machine. The grind size and amount settings were optimized in order to achieve a water pressure recommended for the machine, i.e. 5-20 bar pressure. A single-shot calibrated dose of ground roasted coffee beans (7.5-8 g) was delivered into the basket of the portafilter, and after tamping and trimming the material in the basket, the portafilter was inserted into the group head of the machine. The coffee was then extracted according to the machine specifications for pressurized brewing. The volume of the single shot coffee extract was about 25 mL. The coffee extract was shaken for three days at ambient temperature (23-28° C.) before filtering it under vacuum through an HA filter with a porosity of 0.45 μm (Millipore Corp., Bedford, USA).

If required, a carbohydrate-hydrolyzing enzyme may be used to mitigate against sedimentation of carbohydrate from the coffee extract. Accordingly, the coffee extract (25 mL) may be supplemented with 500 μL of a solution in sodium acetate buffer (pH 5) of a carbohydrate-hydrolyzing enzyme containing at least 0.3 mg active enzyme, shaken for three days at a temperature between 35 and 50° C., and filtered under vacuum through an HA filter with a porosity of 0.45 μm (Millipore Corp., Bedford, USA).

### Example 3

### Analysis of Coffee Extract by High-Performance Liquid Chromatography (HPLC)

Analysis was carried out using a Hewlett-Packard Series 1100 HPLC instrument equipped with a variable wavelength detector, a C18 reverse phase column (InsertSustain, 15 cm×4.6 mm i.d., particle size 5 μm) and an automatic injection system. The mobile phase was a gradient system of water and methanol, with a flow rate of 1 mL/min. The injection volume was 20 μL. Caffeine content was determined by measuring the absorbance at 274 nm. For quantification, an external standard method was used. Standard solutions containing known quantities of caffeine were prepared (1.25-20 mg/L) and used to determine the retention time and content of caffeine in the extracts. The caffeine concentration in the concentrated Soxhlet extract (Example 1) and in the espresso coffee machine extract (Example 2) were found to be 3.1 mg/mL and 3.4 mg/mL, respectively.

### Example 4

### Production of Caffeine Nasal Spray

0.01M Phosphate buffered saline (PBS, pH 7.4 at 25° C.), containing 0.276 M NaCl and 0.0054 M KCl, was prepared according to the manufacturer's instruction (Sigma-Aldrich, St Louis, USA), by dissolving one tablet into 100 mL deionized water. In parallel, 15 mg hypromellose (Sigma-Aldrich) was added to 5.93 mL pure water and stirred slowly for 24 hours at ambient temperature to complete dissolution. Accurately weighed 300 mg caffeine (Scharlab S.L., Barcelona, Spain) and 15 mg potassium sorbate (Sigma-Aldrich) were dissolved in 5.92 mL PBS at 50° C. in a 50-mL container, using power stirring (a vortex mixer), for 5-10 minutes. The concentrated coffee extract (3 mL), as resulted from the operations described either in Example 1 or in Example 2, and 0.15 mL glycerol (Sigma-Aldrich) were added and mixed well in a vortex mixer until the resulting solution became clear. The solution was then transferred into a spray bottle suitable to enclose 15 mL fluid. At this stage, the concentration of PBS achieved 0.01 M, i.e. 0.138M NaCl and 0.0027M KCl. The procedure was performed at room temperature. Based on the caffeine content of the coffee concentrated extract and the supplementary caffeine, the final concentration of caffeine in the nasal spray was 2.07% w/v. Generally, concentrations of approximately 2% w/v caffeine are prepared using this method, which correlates with the maximum solubility of caffeine in water under ambient conditions. Table 1 summarizes the composition of the spray as formulated in Example 4.

TABLE 1
Composition of the caffeine nasal spray

| | Volume/amount | | | | Concentration | |
|---|---|---|---|---|---|---|
| | in 15 mL | | Volume/amount | | in the | |
| Ingredient | (per bottle) | | in 100 mL | | spray mixture | |
| Caffeine | 300 | mg | 2 | g | 2% | w/v |
| Potassium sorbate | 15 | mg | 100 | mg | 0.1% | w/v |
| Coffee extract | 3 | mL | 20 | mL | 20% | v/v |
| (caffeine content) | (10 | mg) | (67 | mg) | (0.07% | w/v) |
| Glycerol | 0.15 | mL | 1 | mL | 1% | v/v |
| Hypromellose | 15 | mg | 100 | mg | 0.1% | w/v |
| PBS | 11.85 | mL | 79 | mL | 79% | v/v |

After introducing the caffeine solution of Example 4 into the spray bottle, and sealing, the caffeine nasal spray may be stored unopened for approximately three months without any adverse effects. Refrigeration is not required. After first opening, typically the caffeine solution should be used or discarded after one month from opening.

As alluded to above, caffeine is known to be absorbed and directly transported though the nasal epithelium and can target the central nervous system through the olfactory pathways [see K. De Pauw et al: “Electro-physiological changes in the brain induced by caffeine or glucose nasal spray”, Psychopharmacology, 234 (2017) 53-62]. Intranasal administration of a composition of the present invention would therefore be expected to provide a central effect.

Administration of 0.1 mL (2 mg) of the composition of Example 4 by nasal spray into each nostril of individual volunteers was considered to be pleasant and generally resulted in relief from coffee cravings.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1. A method of treating or preventing a craving for coffee in an individual, or treating or preventing caffeine withdrawal or a symptom of caffeine withdrawal in an individual, comprising administering to the individual via intranasal delivery an aqueous pharmaceutical composition comprising a natural caffeine extract and at least one pharmaceutically acceptable excipient.

2-3. (canceled)

4. The method according to claim 1, wherein the natural caffeine extract is coffee extract.

5. The method according to claim 1, wherein the method maintains a nil-by-mouth status.

6. The method according to claim 1, wherein the method is carried out prior to a medical procedure.

7. The method according to claim 1, wherein the composition further comprises caffeine or a pharmaceutically acceptable salt, solvate, derivative or metabolite thereof.

8. The method according to claim 7, wherein the composition comprises total caffeine in an amount of about 0.5 to 2.2% w/v

9. The method according to claim 1, wherein the composition is delivered by a nasal spray dispenser.

10. The method according to claim 1, wherein the dispenser is a metered dose device.

11. The method according to claim 10, wherein
the dispenser is configured to dispense a total volume of the composition of 0.05-0.2 mL per actuation.

12. The method according to claim 11, wherein the dispenser delivers approximately 2 mg of caffeine per 0.1 mL actuation.

13. A pharmaceutical composition formulated for nasal administration comprising:
a natural caffeine extract;
at least one pharmaceutically acceptable excipient;
a pharmaceutically acceptable aqueous carrier; and, optionally
caffeine, or a pharmaceutically acceptable salt, solvate, derivative or metabolite thereof.

14. The pharmaceutical composition according to claim 13, wherein the natural caffeine extract is coffee extract.

15. (canceled)

16. The pharmaceutical composition according to claim 13, wherein the composition is adapted for administration by nasal spray.

17. The pharmaceutical composition according to claim 13, wherein the caffeine extract comprises 10-30% v/v of the composition.

18. The pharmaceutical composition according to claim 13, wherein the composition is prepared from anhydrous caffeine at 0.5-2% w/v.

19. The pharmaceutical composition according to claim 13, wherein the composition comprises 0.5-2.1% w/v of total caffeine.

20-24. (canceled)

25. The pharmaceutical composition according to claim 13 comprising:
coffee extract: 8-30% v/v;
additional caffeine: 0.5-2% w/v;
at least one pharmaceutically acceptable excipient; and
saline: 65-90% v/v.

26. The pharmaceutical composition according to claim 13 comprising:
coffee extract: 10-30% v/v;
additional caffeine: 0.5-2% w/v;
potassium sorbate: 0.05-0.2% w/v;
glycerol: 0.5-2% v/v;
hypromellose: 0.01-1% w/v; and
phosphate buffered saline to 100%.

27. A metered dose nasal spray apparatus or device containing a composition according to claim 13.

28. A method of treating or preventing a craving for coffee in an individual, or treating or preventing caffeine withdrawal or a symptom of caffeine withdrawal in an individual, comprising administering to the individual a pharmaceutical composition according to claim 13.

29-30. (canceled)
